SEQUENCE LISTING (40 sequences)

SEQ ID NO: 1 - protein, 17 aa, Homo sapiens
YRVRVTPKEKTGPMKEM

SEQ ID NO: 2 - protein, 19 aa, Homo sapiens
YEKPGSPPREVVPRPRPGV

SEQ ID NO: 3 - protein, 15 aa, Homo sapiens
KNNQKSEPLIGRKKT

SEQ ID NO: 4 - protein, 25 aa, Homo sapiens
DELPQLVTLPHPNLHGPEILDVPST

SEQ ID NO: 5 - protein, 20 aa, Homo sapiens
GEEIQIGHIPREDVDYHLYP

SEQ ID NO: 6 - protein, 5 aa, Homo sapiens
IDAPS

SEQ ID NO: 7 - protein, 5 aa, Homo sapiens
LDVPS

SEQ ID NO: 8 - protein, 8 aa, Homo sapiens
WQPPRARI

SEQ ID NO: 9 - protein, 6 aa, Homo sapiens
HSRNSI

SEQ ID NO: 10 - protein, 15 aa, Homo sapiens
ATETTITISWRTKTE

SEQ ID NO: 11 - protein, 5 aa, Homo sapiens
PHSRN

SEQ ID NO: 12 - protein, 6 aa, Homo sapiens
KLDAPT

SEQ ID NO: 13 - protein, 7 aa, Homo sapiens
EDGIHEL

SEQ ID NO: 14 - protein, 19 aa, Homo sapiens
DYAVLQLHGGRLHFMFDLG

SEQ ID NO: 15 - protein, 19 aa, Homo sapiens
KNSFMALYLSKGRLVFALG

SEQ ID NO: 16 - protein, 12 aa, Homo sapiens
PPFLMLLKGSTR

SEQ ID NO: 17 - protein, 7 aa, Homo sapiens
SIYITRF

SEQ ID NO: 18 - protein, 6 aa, Homo sapiens
IAFQRN

SEQ ID NO: 19 - protein, 5 aa, Homo sapiens
YIGSR

SEQ ID NO: 20 - protein, 6 aa, Homo sapiens
LGTIPG

SEQ ID NO: 21 - protein, 19 aa, Homo sapiens
CSRARKQAASIKVAVSADR

SEQ ID NO: 22 - protein, 20 aa, Homo sapiens
RYVVLPRPVCFEKGMNYTVR

SEQ ID NO: 23 - protein, 13 aa, Homo sapiens
TMKIIPFNRLTIG

SEQ ID NO: 24 - protein, 12 aa, Homo sapiens
HHLGGAKQAGDV

SEQ ID NO: 25 - protein, 5 aa, Homo sapiens
  misc_feature (3)..(4): Xaa can be any naturally occurring amino acid
DLXXL

SEQ ID NO: 26 - protein, 8 aa, Homo sapiens
AEIDGIEL

SEQ ID NO: 27 - protein, 4 aa, Homo sapiens
QIDS

SEQ ID NO: 28 - protein, 6 aa, Homo sapiens
  misc_feature (3)..(3): Xaa can be any naturally occurring amino acid
GFXGER

SEQ ID NO: 29 - protein, 4 aa, Homo sapiens
RGDK

SEQ ID NO: 30 - protein, 5 aa, Homo sapiens
GRGDS

SEQ ID NO: 31 - protein, 6 aa, Homo sapiens
GRGDSP

SEQ ID NO: 32 - protein, 6 aa, Homo sapiens
GRGDNP

SEQ ID NO: 33 - protein, 10 aa, Homo sapiens
RGDSPASSKP

SEQ ID NO: 34 - protein, 4 aa, Homo sapiens
DGEA

SEQ ID NO: 35 - protein, 9 aa, Homo sapiens
CDPGYIGSR

SEQ ID NO: 36 - protein, 5 aa, Homo sapiens
IKVAV

SEQ ID NO: 37 - protein, 10 aa, Homo sapiens
RNIAEIIKDI

SEQ ID NO: 38 - protein, 7 aa, Homo sapiens
YFQRYLI

SEQ ID NO: 39 - protein, 5 aa, Homo sapiens
PDSGR

SEQ ID NO: 40 - protein, 4 aa, Homo sapiens
KRSR

(19) United States
(12) Patent Application Publication
(10) Pub. No.: US 2011/0275539 A1
(43) Pub. Date: Nov. 10, 2011

(54) SUBSTRATE FOR SELECTING AND SPECIFICALLY INFLUENCING THE FUNCTION OF CELLS

(75) Inventors: Joachim P. Spatz, Stuttgart (DE); Nadine Perschmann, Lauffen (DE); Ann-Kathrin Wandner, Ludwigsburg (DE); Roberto Fiammengo, Stuttgart (DE)

(73) Assignee: Max-Planck-Gesellschaft zur Foerderung der Wissenschaften e.V., Muenchen (DE)

(21) Appl. No.: 13/133,087

(22) PCT Filed: Dec. 7, 2009
(86) PCT No.: PCT/EP2009/008723

§ 371 Date: Jul. 15, 2011

(30) Foreign Application Priority Data
Dec. 8, 2008 (DE) 10 2008 060 991.9

(51) Int. Cl. C40B 30/06; C40B 40/00; C12P 21/00; C12Q 1/02; C12Q 1/04; C12N 5/071; C40B 60/12

(52) U.S. Cl. 506/10; 435/375; 506/13; 506/39; 435/29; 435/34; 435/70.3

(57) ABSTRACT

The invention relates to a method and to a substrate for selecting and specifically influencing the function of cells by the adhesion thereof to substrate surfaces having prescribed properties. Said substrates comprise various surface regions each representing a condition affecting the cell adhesion and/or cell function, and said conditions are determined by a geometric property and/or a mechanical property or a combination of a geometric property and/or a mechanical property with a chemical property of each surface region. The invention further relates to analysis devices and to analysis methods using said substrates for identifying and selecting particular cell types, for identifying suitable substrate conditions for affecting a particular cell function or particular cell type or for identifying disease states characterized by a change in the cell type or cell function.

The invention relates to methods and substrates for specifically influencing the function of cells by adhering them to substrate surfaces with predetermined characteristics. The invention also relates to analysis devices and analysis methods using these substrates for identifying and selecting specific cell types, for identifying suitable substrate conditions for influencing a specific cell function or specific cell types or for identifying disease states which are characterized by a change in cell type or cell function.

It is known that the growth and development of biological cells are substantially determined by their spatial environment in natural tissues and their contact with signal molecules in the extracellular matrix (ECM). In the context of wide-ranging research in this field, a large number of such signal molecules and corresponding receptors of various cells have been investigated and their structure elucidated.

Approaches to artificially synthesizing a matrix for regulating cell cultures in vitro are very frequently based on polymeric and inorganic scaffolds. The objective here is generally for the polymeric or inorganic scaffold to provide physical signals to predetermine cell orientation, cell migration and cell propagation. The pores of the scaffold here provide the cell system with sufficient space to define the tissue structure after a certain culture period. Traditional polymer systems are for example polytetrafluoroethylene, silicones or polyethylene. Bioactive glass, ceramics or calcium phosphates may be used as inorganic scaffolds.

As a consequence of recent findings in the field of signal molecules and the interaction thereof with different cell types, matrices comprising a bioactive component are increasingly being developed. Incorporating specific signal molecules, such as for example growth and differentiation factors, into an inert polymer matrix permits very much better control of cell adhesion in contact with these signal molecules (Sakiyama-Elbert and Hubell (2001), Annu. Rev. Mater. Res. 31:183-201).

Such bioactive matrices are of particular interest in the field of implant technology, where the attempt is being made, for example, to achieve better integration of the implants by functionalizing the interfaces to the tissue with signal factors in order to promote in vitro or in vivo colonization with desired cell types.

However, artificial matrices known in the prior art take account of only some of the factors which, under natural conditions, determine interaction between the extracellular matrix and various cell types.

An awareness of further factors and matrix characteristics which determine the interaction with various cell types and indirectly influence functions of these cells would be of major benefit in identifying and selecting specific cell types and for methods for specifically influencing one or more cell functions of target cells.

One object of the present invention is thus to provide new methods and tools with which the function of cells may be specifically influenced by adhering them to substrate surfaces and/or specific cell types may be selected and identified. A closely related further object is to identify suitable substrate conditions for influencing a specific cell function or specific cell types.

The present invention is based on the surprising recognition that not only the chemical characteristics, for example presence of cell ligands and/or signal molecules, but also the mechanical characteristics of a substrate, for example the hardness, strength or rigidity of the substrate, enabling of mechanical stimulation of cells by the substrate, and the geometric characteristics, for example the spatial arrangement of cell binding sites, of a substrate not only have a substantial influence on cell adhesion (Discher et al. (2005), Science 320:1139-1143; Arnold et al. (2004), Chemphyschem. 5:383-388) but also in many cases have a direct and specific influence on certain cell functions of adhering cells.

On the basis of these findings, in order to achieve the above objects, one aspect of the present invention provides substrates for binding cells as claimed in claim 1, in which the substrates comprise different surface domains which in each case represent a condition with an influence on cell adhesion and/or cell function. By combining various substrate parameters essential to cell adhesion and/or cell function in a surface domain and purposefully varying them in other surface domains, it is possible rapidly and efficiently to identify suitable or optimized substrate conditions for specific cell types or specific cell functions. Rapid selection and identification of specific cell types also becomes possible in this manner. These substrates are therefore particularly suitable for high-throughput screening of cells, for example as claimed in claim 35.

In more specific embodiments, such substrates are a component of a biomaterial chip as claimed in claim 19 or an analysis device as claimed in claim 21.

The invention also encompasses the use of the above substrates, chips and analysis devices in various, in particular medical, applications as claimed in claims 25-34.

A second aspect of the present invention provides a method for purposefully influencing protein synthesis of target cells on a substrate as claimed in claim 36, with which the synthesis of desired proteins is induced or influenced by the arrangement of cell ligands in a predetermined spacing on the substrate.

A third aspect of present invention provides a method for selecting and/or identifying cells as claimed in claim 41, in which the specific response of cells, which are located on a substrate, to mechanical stimulation is recorded and evaluated.

Further specific and/or preferred embodiments of the invention are the subject matter of the dependent claims.

In the substrates of the present invention, the conditions which influence cell adhesion and/or cell function are typically determined at least by a geometric characteristic and/or a mechanical characteristic or a combination of a geometric characteristic and/or a mechanical characteristic with a chemical characteristic of the respective surface domain. It is, however, entirely possible for still further characteristics, for example the timing and duration of contact between substrate and cells, likewise to play an important role in influencing cell adhesion and/or cell function. The present invention accordingly likewise encompasses any desired combinations of further characteristics with the chemical, mechanical and geometric characteristics explained here in greater detail.

The chemical characteristic of the substrate is first of all determined by the molecular structure of the inorganic or organic substrate. The substrate may be, for example, glass, metal or a plastics material. Nanostructure domains having a predetermined spacing of nanostructures may be provided on this base substrate. Such nanostructures are produced, for example, by applying gold nanoclusters of a desired size and with a desired spacing on a substrate. Preferably, the chemical characteristic of a desired surface domain or plurality of surface domains is predetermined or largely determined by functionalizing the base substrate and/or the nanostructures with specific cell ligands, in particular molecules of the extracellular matrix (ECM) in natural tissues or fragments thereof.

A non-limiting selection of suitable ligands is indicated below. A person skilled in the art will however immediately realize that variations of these molecules and any desired other molecules having specific binding properties for certain target cells may likewise be used.

The cell ligands are typically selected from molecules which bind to cell adhesion receptors (CAM) of cells. More specifically, these are molecules which bind to the cell adhesion receptors of the cadherin, immunoglobulin superfamily (Ig-CAMS), selectin and integrin groups, in particular to integrins.

Still more specifically, the ligands are selected from fibronectin, laminin, fibrinogen, tenascin, VCAM-1, MadCAM-1, collagen or a fragment thereof which binds specifically to cell adhesion receptors, in particular integrins, or a derivative thereof which binds specifically to cell adhesion receptors.

Table 1 provides a non-limiting list of suitable specifically binding amino acid sequences.

The geometric characteristic of the substrate typically involves the arrangement of contact points for the cells, in particular functionalized with cell ligands, in a predetermined spacing on the substrate.

In a more specific embodiment, the arrangement of contact points or cell ligands constitutes a nanostructure.

The term “nanostructure”, as used here, denotes an arrangement of nanometer-sized islands, hereinafter denoted “nanostructure domains”, which may serve as contact points and may be selectively surface-modified with further molecules, for example cell ligands. The size of the islands should be no greater than a countable quantity of molecules which interacts with the surface of the cells. A desirable size of an island is that which, due to the size of the island, enables just one individual molecule to interact.

Island diameters in the range of smaller than 100 nm, in particular less than 20 nm, for example less than 10 nm, are here of particular interest and are preferably used.

In order to produce a desired topology, it must be possible to adjust the spacing of the islands in ranges of between 1 and 1000 nanometers, in particular 1-300 nm, for example 1-200 nm or preferably 1-100 nm, flexibly and to an accuracy of 1-2 nanometers. In this manner, it is for example possible to provide a fit with the scale of length predetermined along cell membranes (lock-and-key principle), such that selective attachment of specific cells at the interfaces can be achieved. Such attachment may involve single or multiple attachment of an individual cell. In particular in the presence of flow or, more generally, externally applied forces, multiple attachment has the advantage of retaining the cells distinctly more strongly at the interface than would be possible with single attachment, thus optionally facilitating the separation thereof from mixed media.

Conventional structuring methods for surfaces such as photolithography or electron beam lithography or structuring methods based thereon are suitable only to a limited extent for the molecularly accurate positioning of nanostructures, as used in the present invention, on large-area interfaces of any desired shape. In contrast, due to the nature of the structural ordering which arises spontaneously, structuring methods based on molecular self-organization are well suited to the production of the nanostructures used according to the invention. The technique of block copolymer micelle nanolithography may particularly advantageously be used to this end (R. Glass, M. Möller, J. P. Spatz, Nanotechnology 2003, 14 (10), 1153-1160, Arnold, M. Cavalcanti-Adam, R. Glass, J. Blummel, W. Eck, M. Kantlehner, H. Kessler, and J. P. Spatz. (2004) Chemphyschem. 5:383-8, DE 199 52018.6; DE 19747813.1; DE 29747815.8; DE 19747816.6).

In this manner, e.g. gold nanoclusters of a desired size and desired spacing may be produced on a substrate. These nanostructures are typically quasi-hexagonally ordered. Substrates which may be considered are in principle any materials on which the desired nanostructures may be formed. A few suitable, but non-limiting examples are glass, silicon dioxide and plastics surfaces, including hydrogels.

In one more specific embodiment of the present invention, the nanostructure domains form gradients of at least one of the geometric (e.g. spacing and size of the domains) and chemical characteristics (e.g. nature of functionalization). These gradients may also be formed by a sequence of spatially separated domains (see FIG. 6).

In one typical embodiment of the invention, the mechanical characteristic comprises the hardness or rigidity of the substrate and/or the viscous, elastic or viscoelastic characteristics thereof. Hardness or rigidity, expressed as Young's modulus, may here be varied over a range from 0.1 kPa to 100 MPa. Alternatively or additionally, the mechanical characteristic may comprise mechanical stimulation of the cells by the substrate.

Mechanical characteristics are varied by using for example resilient plastics such as polydimethylsiloxane (PDMS) or hydrogels, preferably PEO-based, particularly preferably polyethylene glycol diacrylate (PEGDA) hydrogels, as the base substrates. Different rigidities may be provided by using different hydrogels, for example polyethylene glycol diacrylate hydrogels (PEGDA). If PEGs of various molecular weights and different weight percentages (concentrations) are used, these offer a wide Young's modulus range (ranging from MPa to kPa). Nanostructures as described above may likewise be transferred onto these base substrates (e.g. as described in DE 10 2004 043 908 A1). These gold nanostructures, which may exhibit different particle densities and thus variable particle spacing (in the form of a gradient or of precise, homogeneous domains) and are purposefully adjustable in this respect, act, once transferred onto the hydrogel, as binding sites for various ligands and/or functional groups, such as for example thiol groups.

One method in principle for producing hydrogel-based substrates according to the invention is described in greater detail below and in Example 1. It will, however, be apparent to a person skilled in the art that substrates which are likewise suitable may be produced by using alternative reagents, which perform the same function, and by appropriately varying reaction parameters. In the light of the general technical teaching and examples disclosed herein, such modifications are within the ability of an average person skilled in the art and require no more than routine testing.

In order to attach the PEG hydrogel when producing such a hydrogel substrate, a surface (e.g. glass) of a base substrate is first of all activated/hydroxylated and functionalized with a compound which for example contains an unsaturated function, for example allyltriethoxysilane (FIG. 1a). The purpose of the unsaturated function is here subsequently to crosslink with the hydrogel during the polymerization process.

Nanostructuring of the hydrogel is brought about by a transfer process. To this end, the nanostructure is first of all applied onto a glass surface by means of a diblock copolymer micelle technique (e.g. similar to that described in DE 199 52018.6; DE 19747813.1; DE 29747815.8; DE 19747816.6) and then transferred onto the hydrogel using a linker, for example a propenethiol or N,N′-bis(acryloyl)cystamine linker. The unsaturated end of the linker here serves to form covalent bonds during polymerization of the hydrogel.

The entire substrate is then prepared by simultaneously transferring the gold structure and attaching the hydrogel during the polymerization process. The surfaces in question here serve as a flow cell (FIG. 1b), whereby the polymer solution may be filled in bubble-free manner between the two surfaces. After subsequent irradiation with UV light, for example with a wavelength of 365 nm, for polymerization the hydrogel is immersed in water, whereby the gel absorbs water and is gently detached from the upper glass. Using this method, it is possible to bind the various hydrogels to even large areas (e.g. 8 cm×12 cm).

In this manner, by using a plurality of small flow cells with different nanostructures, it is possible to produce a substrate which simultaneously comprises hydrogels of differing levels of firmness and varying gold particle spacing. Different ligands may then be attached to these gold particles of the nanostructured hydrogels, so adding a further variable parameter.

In addition to the previously stated parameters, the PEG hydrogel may moreover be functionalized entirely or in part with a carboxylic acid. This laterally controlled functionalization of the surface of the gel proceeds by a transfer process. To this end, a carboxylic acid, preferably a long-chain, polyunsaturated carboxylic acid (fatty acid, for example linolenic acid) is first of all applied onto a hydrophilic glass. This procedure is associated with a high lateral precision and permits functionalization in the form of a microstructure (FIG. 1c), which may also be transferred onto the gel. During the polymerization process, the unsaturated end of the acid is then crosslinked with the PEG hydrogel, whereby the carboxylic acid is covalently bonded to the surface of the gel (FIG. 1d).

This functionalization likewise has a considerable impact on the growth behavior of cells on the corresponding substrate. The left-hand field of FIG. 2 clearly shows that the cells grow exclusively on the side of the hydrogel functionalized with carboxylic acid. The unfunctionalized side has a passivating action on cells. The right-hand field shows the attachment of the fluorescent dye Oregon Green 488 cadaverine by means of EDC/NHS activation of the carboxyl function.

A preferred embodiment of the invention provides a substrate which comprises at least 3 different surface domains having at least 3 different conditions which influence cell adhesion and/or cell function.

This substrate is preferably distinguished in that the at least 3 different conditions which influence cell adhesion and/or cell function comprise the functionalization of at least one surface domain with specific cell ligands, the geometric arrangement of cell ligands in at least one surface domain, and substrate hardness or substrate rigidity in at least one surface domain. A combination of two or more characteristics in one or more of these surface domains is also possible and generally preferred (FIG. 3).

These parameters may be varied in different combinations in precise domains of the substrate and be adapted for specific applications and comprise three of the most important focal points of observation with regard to influencing adherent cells.

In one specific embodiment, the substrate has a three-dimensional structure. Such a structure may be, for example, a tube or microtube with a diameter of a few to several 100 micrometers. Such tubes may be composed of various plastics and hydrogels, for example based on polyethylene glycol, and also comprise nanostructures, for example produced as described above.

In another specific embodiment, the different surface domains of the substrate are spatially separated from one another by barriers. For example, the different surface domains may be located in separate chambers of the substrate.

The present invention also comprises carriers which comprise two or more of the substrates according to the invention in a three-dimensional arrangement. For example, two identical or different substrates, for example bearing different cell ligands, may be placed against one another. The ideal spacing may be produced, for example, by means of a frame (e.g. made of Teflon) of a specific thickness between the two substrates. Medium may here be exchanged by gently pumping through fresh medium.

A preferred embodiment of the invention relates to a biomaterial chip which comprises at least one substrate according to the invention and is composed of different separate chambers which represent different but specific, conditions which influence cell adhesion and/or cell function as explained above and permit separated culturing of cells in each chamber. Such a biomaterial chip preferably comprises at least 16 chambers.

The present invention also provides an analysis device comprising
a) a substrate, a carrier or a biomaterial chip as defined above,
b) a sample holder, in which the substrate or carrier or biomaterial chip is arranged,
c) a measurement device for detecting at least one cell-specific analytical parameter and
d) an evaluation device.

The measurement device generally comprises a direct or inverted optical microscope and preferably a device for digital image processing.

The cell-specific analytical parameter is typically the cell count, the cell shape or the presence of a marker, in particular a fluorescence marker, for example for adhesion molecules or certain specific proteins, for example for cell differentiation, or other specific molecules, for example nucleic acids or membrane components, but is not limited thereto. Further suitable parameters will be immediately apparent to a person skilled in the relevant art as a function of the cells investigated and the culture conditions and be determinable using standard methods.

The above-described substrates, biomaterial chips or analysis devices may be used, for example, for identifying suitable substrate conditions for a specific cell system or a specific cell function. In one specific embodiment, this specific cell function is the synthesis of specific proteins.

The substrates, chips and analysis devices according to the invention are particularly suitable for carrying out screening with large numbers of samples and/or many different substrate conditions (“high-throughput screening”; HTS). By combining various substrate parameters essential for cell adhesion and/or cell function in one or more surface domain(s) and purposefully varying them in further surface domains, it is possible rapidly and efficiently to identify suitable or optimized substrate conditions for specific cell types or specific cell functions. Rapid selection and identification of specific cell types also becomes possible in this manner.

The cells investigated are limited neither in terms of cell type nor nature of the species. Both prokaryotic and eukaryotic cells may be investigated. The cells are preferably those of a vertebrate, in particular of a mammal, particularly preferably of a human. In one specific embodiment, the investigated cells are stem cells or differentiated cells derived therefrom.

The substrates, chips and analysis devices according to the invention have wide-ranging potential applications in many fields of biology, biochemistry and medicine, including medical diagnostics. Specific fields of application are for example investigations in immunology and allergology. One specific example is the identification of substrate conditions for triggering allergic reactions of T or mast cells.

A further potential application relates to promoting or investigating selective cell colonization of interfaces, in particular in cardiology or implant technology. The result of this investigation may be, for example, the identification of suitable or optimized matrix characteristics for implants in different regions of the body, for example bone, ear etc.

The above-described substrates, biomaterial chips or analysis devices may also be used for selecting or identifying cells. One associated application is the identification of disease states which are characterized by a change in cell type, for example cancer or malaria.

A further aspect of the present invention relates to a method for influencing the protein synthesis of target cells comprising
a) providing a substrate for binding cells to the surface of this substrate, the substrate comprising at least one surface domain which is functionalized with cell ligands which are arranged on the substrate in a predetermined spacing;
b) applying the target cells onto the substrate;
c) culturing the target cells on the substrate, synthesis of desired proteins being induced or influenced by the arrangement of cell ligands in a predetermined spacing on the substrate.

This method may furthermore involve additionally providing a specific mechanical characteristic of the functionalized surface domain which likewise influences cell function. This mechanical characteristic may be provided, for example, by predetermining a specific rigidity or hardness of the substrate or by providing mechanical stimulation of adhering cells.

A further aspect of the present invention exploits the recognition already mentioned above that different cells respond very differently to mechanical stimulation by a substrate on which they are located. Said aspect accordingly relates to a method for selecting and/or identifying cells which comprises:
a) providing a substrate for binding cells to the surface of this substrate, the substrate comprising at least one surface domain which is capable of mechanically stimulating the cells;
b) applying the target cells onto the substrate;
c) mechanically stimulating the target cells on the substrate;
d) recording the response of the cells to the stimulation;
e) evaluating the response of the cells and optionally making a comparison with reference values and consequently identifying cells of a specific cell type and/or a specific origin.

### DESCRIPTION OF THE FIGURES

FIG. 1a is a schematic diagram of the functionalization of a base substrate (e.g. glass) with allyltriethoxysilane for attachment to a hydrogel;

FIG. 1b is a schematic diagram of the attachment of the hydrogel and the transfer of a gold nanostructure onto the hydrogel;

FIG. 1c shows the microstructuring of a hydrophilic glass with carboxylic acid for subsequent transfer onto a hydrogel;

FIG. 1d is a schematic diagram of the functionalization of the attached hydrogel by the carboxylic acid.

FIG. 2 shows various hydrogels attached to glass. The right-hand field illustrates the transfer of the gold nanostructure by “electroless deposition”, by which the gold particles are enlarged.

FIG. 3 is a schematic diagram of a combination of three variable parameters on a substrate with different surface domains.

FIG. 4 shows a comparison of the growth of cells on a hydrogel functionalized with carboxylic acid relative to an unfunctionalized hydrogel.

FIG. 5 shows contact angle measurements of surfaces with and without carboxylic acid functionalization.

FIG. 6 is a schematic diagram of an embodiment of a substrate with a plurality of spatially separate surface domains, in which two parameters, namely the nature of the ligand (biomolecules 1-4) and the spacing between the ligands are systematically varied.

FIG. 7 shows the different synthesis activity of 3T3 fibroblasts with regard to the protein fibronectin as a function of the structure of the substrate surface, detected by means of gel electrophoresis of the corresponding mRNA.

FIG. 8 shows a bar chart of the different synthesis activity of mouse osteoblasts with regard to the protein vinculin as a function of the structure of the substrate surface.

TABLE 1

| | | Recep- |
|---|---|---|
| Amino acid sequence | Receptors | tors |
| Fibronectin peptides | | |
| YRVRVTPKEKTGPMKEM (C-terminal | A₄β₁ | |
| heparin-binding domain) | | |
| YEKPGSPPREVVPRPRPGV (C-terminal | A₄β₁ | |
| heparin-binding domain) | | |
| KNNQKSEPLIGRKKT (C-terminal | A₄β₁ | |
| heparin-binding domain) | | |
| DELPQLVTLPHPNLHGPEILDVPST | A₄β₁ | α₄β₇ |
| (IIICS, CS1) | | |
| GEEIQIGHIPREDVDYHLYP | A₄β₁ | α₄β₇ |
| (IIICS, CS5) | | |
| IDAPS (IIICS) | A₄β₁ | |
| LDVPS (IIICS) | A₄β₁ | α₄β₇ |
| WQPPRARI | A₄β₁ | |
| HSRNSI (cell-binding domain) | α_{iib}β3 | |
| ATETTITISWRTKTE (C-terminal | α_{iib}β3 | |
| heparin-binding domain) | | |
| PHSRN (Repeat III 9) | A₅β₁ | |
| KLDAPT (Repeat III5) | A₄β₁ | α₄β₇ |
| EDGIHEL | A₄β₁ | α₉β₁ |
| Laminin peptides | | |
| DYAVLQLHGGRLHFMFDLG (LG4 | A₂β₁ | |
| module, laminin alpha1 chain | | |
| hEF-1) | | |
| KNSFMALYLSKGRLVFALG (LG4 | syndecan-2 | |
| module, laminin alpha3 | | |
| PPFLMLLKGSTR (LG3 domain, | A₃β₁ | |
| laminin-5 alpha3 chain) | | |
| SIYITRF (LG1 domain, laminin | A₆β₁ | |
| alpha1 chain) | | |
| IAFQRN (LG2 domain, laminin | A₆β₁ | |
| alpha1 chain) | | |
| YIGSR | 67 kD protein | |
| | receptor | |
| LGTIPG | 67 kD protein | |
| | receptor | |
| CSRARKQAASIKVAVSADR | 32 kD Mac-2P | |
| | protein, 67 | |
| | kD receptor | |
| RYVVLPRPVCFEKGMNYTVR | heparin | |
| Fibrinogen peptides | | |
| ganma 190-202 or P1 | A_{M}β_{2(Mac-1)} | |
| ganma 228-253 in P1 | | |
| ganma 377-395 or P2 | A_{M}β_{2(Mac-1)} | |
| ganma 383-395 or | A_{M}β₂ | αₓβ₂ |
| P2C/TMKIIPFNRLTIG | | |
| HHLGGAKQAGDV (gamma chain) | α_{IIb}β₃ | |
| GPR (alpha chain) | Axβ2 | |
| ganma 373-385 | α_{IIb}β₃ | |
| Tenascin | | |
| DLXXL | Aᵥβ₆ | |
| AEIDGIEL | A₉β₁ | |
| VCAM-1 | | |
| QIDS | A₄β₁ | |
| MadCAM-1 | | |
| LDT | A₄β₇ | |
| Collagen | | |
| GFOGER | A₁β₁ | α₂β₁ |
| Further peptides: | | |
| cyclo(RGDfK) | αᵥβ₃ | αᵥβ₅ |
| GRGDS | αᵥβ₃ | α₅β₁ |
| GRGDSP | A₅β₁ | |
| GRGDNP | A₅β₁ | |
| RGDSPASSKP | A₅β₁ | |
| Collagen | | |
| DGEA | A₂β₁ | |
| Laminin-derived peptides | | |
| (binding to nerve cells) | | |
| CDPGYIGSR | ? | |
| IKVAV | ? | |
| RNIAEIIKDI | ? | |
| YFQRYLI | ? | |
| PDSGR | ? | |
| Heparin-binding sequence | | |
| KRSR | | |

CAM = “Cell Adhesion Molecule”

The following examples are intended to explain the invention in greater detail but without restricting it thereto.

### EXAMPLE 1

### Production of a Hydrogel Substrate

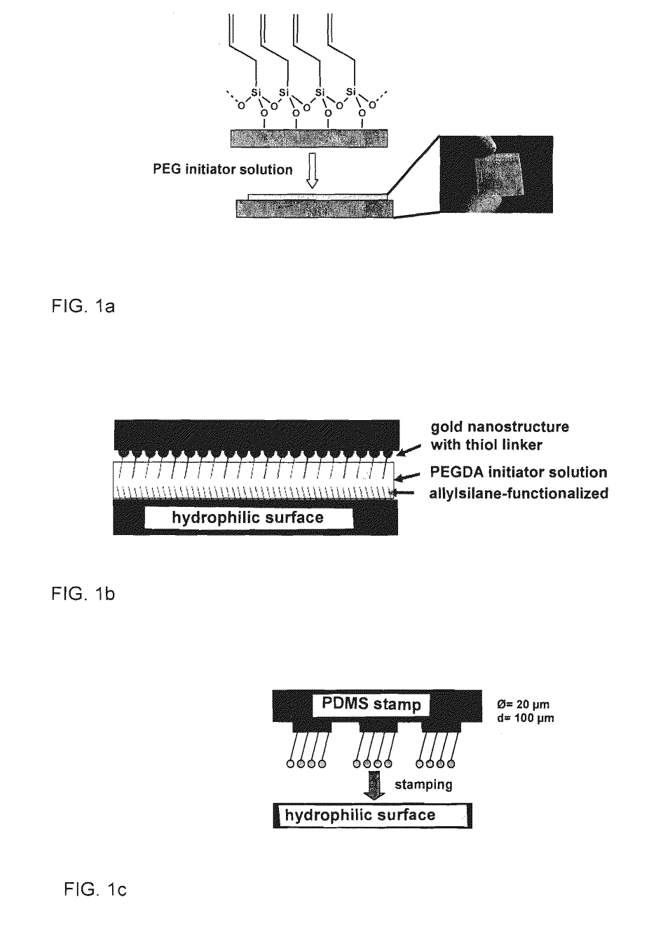

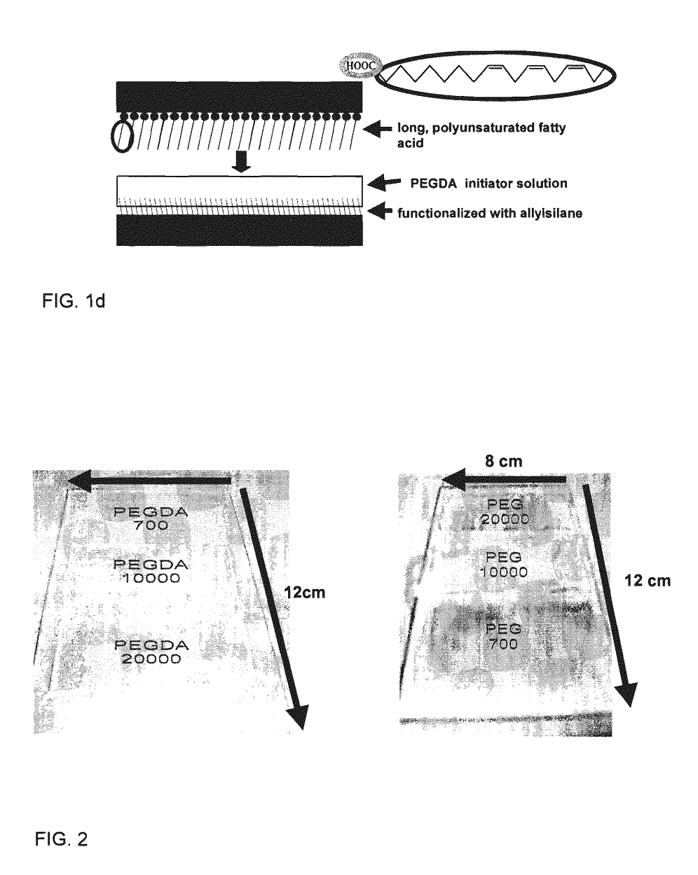

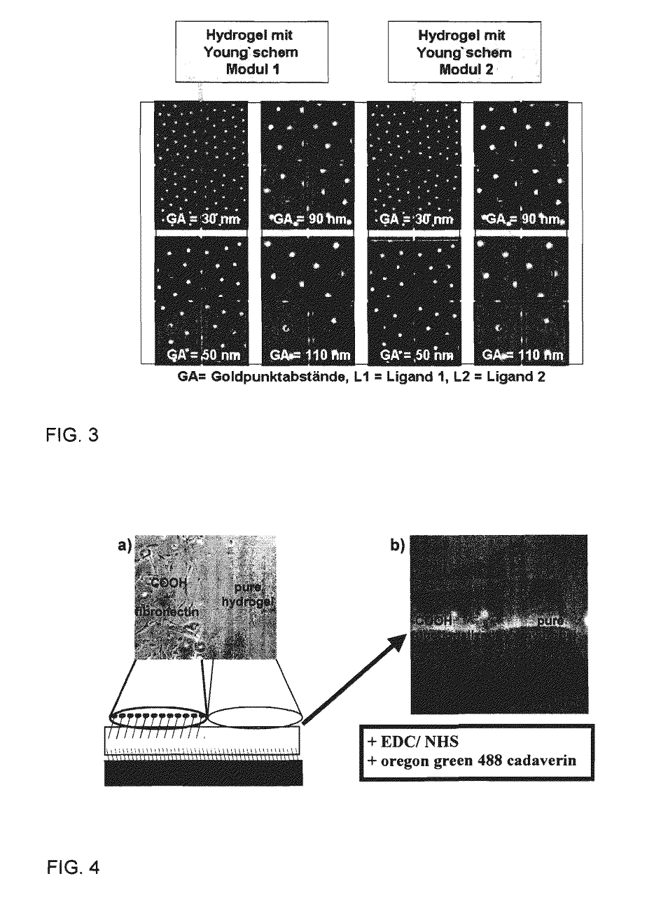

In order to attach the PEG hydrogel, a surface (glass) is first of all activated/hydroxylated and functionalized with allyltriethoxysilane (FIG. 1a). The purpose of the unsaturated function is here subsequently to crosslink with the hydrogel during the polymerization process.

The hydrogel is nanostructured by a transfer process. To this end, the nanostructure is first of all applied by means of a diblock copolymer micelle technique as previously described onto a glass surface and then transferred onto the hydrogel using a propenethiol or N,N′-bis(acryloyl)cystamine linker. The unsaturated end of the linker here serves to form covalent bonds during polymerization of the hydrogel.

The entire substrate is then prepared by simultaneously transferring the gold structure and attaching the hydrogel during the polymerization process. The surfaces in question here serve as a flow cell (FIG. 1b), whereby the polymer solution may be filled in bubble-free manner between the two surfaces. After subsequent irradiation with UV light with a wavelength of 365 nm, the hydrogel is immersed in water, whereby the gel absorbs water and is gently detached from the upper glass.

By using a plurality of small flow cells with different nanostructures, it is possible to produce a substrate which simultaneously comprises hydrogels of differing levels of firmness and varying gold particle spacing. Various ligands may now be bound to these gold particles of the nanostructured hydrogels.

Such a PEG hydrogel may furthermore be functionalized entirely or in part with a carboxylic acid. This laterally controlled functionalization of the surface of the gel proceeds by a transfer process. To this end, a long-chain, polyunsaturated carboxylic acid (fatty acid, for example linolenic acid) is first of all applied onto a hydrophilic glass (FIG. 1c). During the polymerization process, the unsaturated end of the acid is then crosslinked with the PEG hydrogel, whereby the carboxylic acid is covalently bonded to the surface of the gel (FIG. 1d).

FIG. 4 clearly shows that the cells grow exclusively on the side of the hydrogel functionalized with carboxylic acid. The unfunctionalized side has a passivating action on cells. The right-hand field shows the attachment of the fluorescent dye Oregon Green 488 cadaverine by means of EDC/NHS-activation of the carboxyl function.

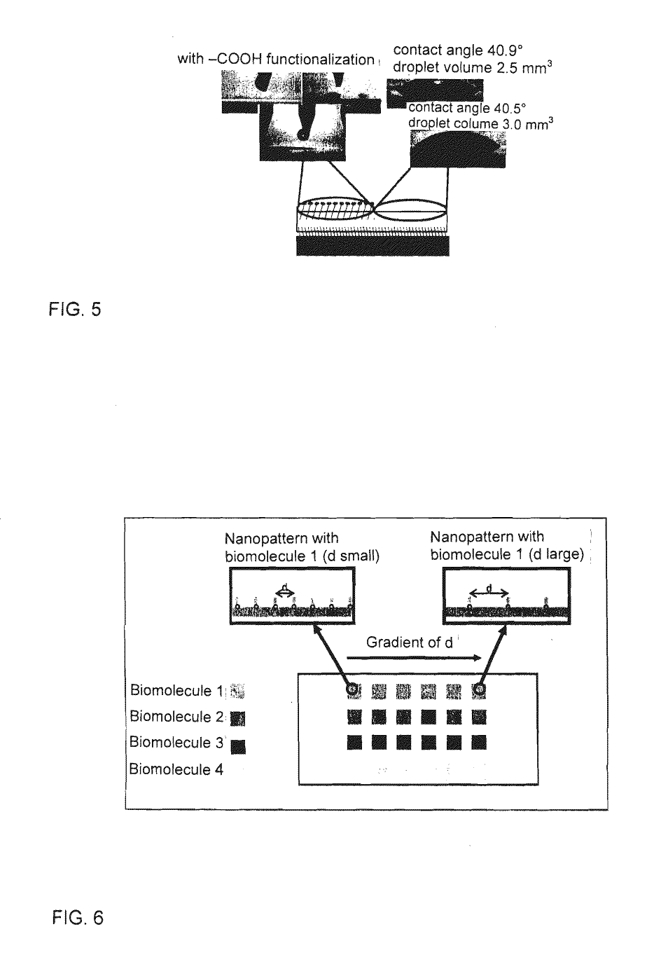

The surface characteristics were moreover also verified by contact angle measurements. These measurements revealed the carboxylic acid functionalized surfaces to be of a distinctly more highly hydrophilic nature. No droplet could be formed here in order to measure the contact angle (FIG. 5).

### EXAMPLE 2

### Different Synthesis Activity of 3T3 Fibroblasts with Regard to the Protein Fibronectin as a Function of the Structure of the Substrate Surface

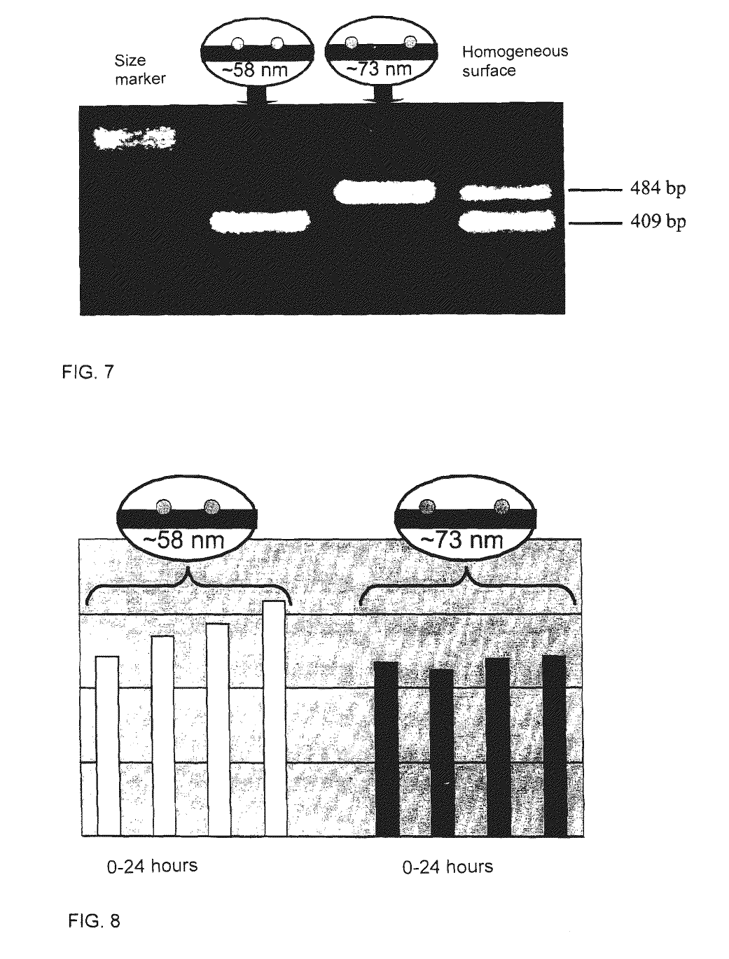

3T3 fibroblasts were applied onto glass substrates which had an arrangement of specific cell ligands, C-(-RGDfK-)thiol, on gold nanostructures with different spacing or a homogeneous surface. These fibroblasts synthesize two different types of fibronectin which differ with regard to molecular weight. On a homogeneous surface, both types are synthesized in a virtually identical quantity. In contrast, predetermining and varying a nanostructure may dramatically shift the preference towards one or the other protein type. FIG. 7 shows the gel electrophoresis of the mRNAs which are responsible for synthesis of the two different fibronectins. The results reveal a great variation in expression activity for the two genes as a function of the spacing of the nanostructure domains and thus of the cell ligands (58 nm or 73 nm respectively).

### EXAMPLE 3

### Different Synthesis Activity of Mouse Osteoblasts with Regard to the Protein Vinculin as a Function of the Structure of the Substrate Surface

Mouse osteoblasts were applied onto glass substrates which had an arrangement of specific cell ligands, C-(-RGDfK-)thiol, on gold nanostructures with different spacing or a homogeneous surface. The extent and time profile of vinculin protein synthesis is highly dependent on the nanostructure selected (58 nm spacing as opposed to 73 nm).

FIG. 8 shows a bar chart of the different synthesis activity of mouse osteoblasts with regard to the protein vinculin as a function of the structure of the substrate surface over a 24 h period.

1. A substrate for binding cells to a surface of the substrate, wherein the substrate comprises different surface domains, which in each case represents a condition which influences cell adhesion and/or cell function, each condition being determined by a geometric characteristic and/or a mechanical characteristic or a combination of a geometric characteristic and/or a mechanical characteristic with a chemical characteristic of a particular surface domain.

2. The substrate as claimed in claim 1, wherein the geometric characteristic comprises an arrangement of cell ligands in a predetermined spacing on the substrate.

3. The substrate as claimed in claim 2, wherein the arrangement of the cell ligands represents a nanostructure.

4. The substrate as claimed in claim 1, wherein the mechanical characteristic comprises a hardness or a rigidity of the substrate, measured as Young's modulus.

5. The substrate as claimed in claim 1, wherein the mechanical characteristic comprises mechanical stimulation of the cells.

6. The substrate as claimed in claim 1, wherein the chemical characteristic comprises a functionalization of a surface domain with specific cell ligands comprising molecules of an extracellular matrix (ECM) in natural tissues or fragments thereof.

7. The substrate as claimed in claim 6, wherein the cell ligands are selected from molecules which bind to cell adhesion receptors (CAM) of cells.

8. The substrate as claimed in claim 7, wherein the cell ligands are selected from molecules which bind to cell adhesion receptors of a cadherin, immunoglobulin superfamily (Ig-CAMS), selectin and integrin groups.

9. The substrate as claimed in claim 7, wherein the cell ligands are members selected from the group consisting of fibronectin, laminin, fibrinogen, tenascin, VCAM-1, MadCAM-1, collagen and a fragment thereof which binds specifically to cell adhesion receptors.

10. The substrate as claimed in claim 1, wherein the substrate comprises at least 2 different surface domains having at least 2 different conditions which influence cell adhesion and/or cell function.

11. The substrate as claimed in claim 10, wherein the at least 2 different conditions which influence cell adhesion and/or cell function comprise two members selected from the group consisting of functionalization of at least one surface domain with specific cell ligands, a geometric arrangement of cell ligands in at least one surface domain, and substrate rigidity in at least one surface domain.

12. The substrate as claimed in claim 1, comprising a three-dimensional structure.

13. The substrate as claimed in claim 1, wherein the different surface domains are spatially separated from one another by barriers.

14. The substrate as claimed in claim 13, wherein the different surface domains are located in separate chambers of the substrate.

15. The substrate as claimed in claim 1, wherein one or more surface domain(s) comprise(s) a polyethylene glycol diacrylate (PEGDA) hydrogel of a predetermined rigidity.

16. The substrate as claimed in claim 15, wherein gold nanostructures with a predetermined particle spacing are located on the hydrogel as binding sites for cell ligands.

17. A carrier comprising two or more substrates in a three-dimensional arrangement, wherein each of said substrates is a substrate in accordance with claim 1.

18. The carrier as claimed in claim 17, wherein the substrates are spatially separated from one another.

19. A biomaterial chip comprising at least one substrate as claimed in claim 1, said biomaterial chip comprising various separate chambers which represent different but specific conditions which influence cell adhesion and/or cell function and permit separated culturing of cells in each chamber.

20. The biomaterial chip as claimed in claim 19 comprising at least 16 chambers.

21. An investigational device comprising
a) a substrate as claimed in claim 1, a carrier comprising two or more substrates in a three-dimensional arrangement, wherein each of said substrates is a substrate in accordance with claim 1, or a biomaterial chip comprising at least one substrate as claimed in claim 1, said biomaterial chip comprising various separate chambers which represent different but specific conditions which influence cell adhesion and/or cell function and permit separated culturing of cells in each chamber,
b) a sample holder, in which the substrate or carrier or biomaterial chip is arranged,
c) a measurement device for detecting at least one cell-specific analytical parameter, and
d) an evaluation device.

22. The investigational device as claimed in claim 21, wherein the measurement device comprises a direct or inverted optical microscope.

23. The investigational device as claimed in claim 21, wherein the measurement device comprises a device for digital image processing.

24. The investigational device as claimed in claim 21, wherein the cell-specific analytical parameter is a cell count, a cell shape or a presence of a fluorescence marker, for adhesion molecules, or cell differentiation.

25. A method of using a substrate of claim 1, or a carrier, biomaterial chip or investigational device comprising the substrate for identifying suitable substrate conditions for a specific cell system or a specific cell function.

26. The method as claimed in claim 25, wherein the specific cell function is a synthesis of specific proteins.

27. The method as claimed in claim 25, wherein the cell system comprises stem cells or the cell function is a stem cell function.

28. The method as claimed in claim 25, wherein substrate conditions for triggering allergic reactions of T or mast cells are identified.

29. A method of using a substrate of claim 1, or a carrier, biomaterial chip or investigational device comprising the substrate for identifying suitable matrix characteristics for implants in different regions of a body.

30. A method of using a substrate of claim 1, or a carrier, biomaterial chip or investigational device comprising the substrate for selecting or identifying cells.

31. A method of using a substrate of claim 1, or a carrier, biomaterial chip or investigational device comprising the substrate for identifying a disease state which is characterized by a change in cell type.

32. The use as claimed in claim 31, wherein the disease state is cancer or malaria.

33. A method of using a substrate of claim 1, or a carrier, biomaterial chip or investigational device comprising the substrate for investigations in immunology and allergology.

34. A method of using a substrate of claim 1, or a carrier, biomaterial chip or investigational device comprising the substrate for promoting or investigating selective cell colonization of interfaces in cardiology or implant technology.

35. A method of using a substrate of claim 1, or a carrier, biomaterial chip or investigational device comprising the substrate for high-throughput screening of cells.

36. A method for influencing a protein synthesis of target cells comprising
a) providing a substrate for binding cells to a surface of the substrate, the substrate comprising at least one surface domain which is functionalized with cell ligands comprising molecules of an extracellular matrix (ECM) in natural tissues or fragments thereof, which are arranged on the substrate in a predetermined spacing;
b) applying the target cells onto the substrate;
c) culturing the target cells on the substrate, synthesis of desired proteins being induced or influenced by an arrangement of cell ligands in a predetermined spacing on the substrate.

37. The method as claimed in claim 36, further comprising providing a specific mechanical characteristic of the at least one surface domain which is functionalized, which likewise influences cell function.

38. The method as claimed in claim 37, wherein the mechanical characteristic is provided by predetermining a specific rigidity or hardness of the substrate or by providing mechanical stimulation of adhering cells.

39. A method for influencing a protein synthesis of target cells comprising
a) providing a substrate for binding cells to a surface of the substrate, the substrate comprising at least one surface domain which is functionalized with cell ligands comprising molecules of an extracellular matrix (ECM) in natural tissues or fragments thereof, which are arranged on the substrate in a predetermined spacing;
b) applying the target cells onto the substrate;
c) culturing the target cells on the substrate, synthesis of desired proteins being induced or influenced by an arrangement of cell ligands in a predetermined spacing on the substrate,
wherein the substrate is a substrate as claimed in claim 1.

40. The method as claimed in claim 36, wherein the cells are stem cells.

41. A method for selecting and/or identifying target cells comprising:
a) providing a substrate for binding cells to a surface of the substrate, the substrate comprising at least one surface domain which is capable of mechanically stimulating the cells;
b) applying the target cells onto the substrate;
c) mechanically stimulating the target cells on the substrate;
d) recording a response of the target cells to the stimulation; and
e) evaluating the response of the target cells and optionally making a comparison with reference values and consequently identifying cells of a specific cell type and/or a specific origin.
